(19) United States
(12) Patent Application Publication
(10) Pub. No.: US 2024/0180168 A1
(43) Pub. Date: Jun. 6, 2024

(54) ANTIMICROBIAL COMPOSITIONS OF ESSENTIAL OIL MICELLES

(71) Applicant: SOCPRA SCIENCES ET GENIE S.E.C., Sherbrooke (CA)

(72) Inventors: Jan J. DUBOWSKI, Orléans (CA); Amanpreet SINGH, Sherbrooke (CA); Mohamed Walid HASSEN, Sherbrooke (CA)

(21) Appl. No.: 18/551,285

(22) PCT Filed: Mar. 23, 2022
(86) PCT No.: PCT/CA2022/050436

§ 371 Date: Sep. 19, 2023

(51) Int. Cl. A01N 65/28; A01N 25/04; A01N 65/22; A01P 1/00

(52) U.S. Cl. CPC A01N 65/28; A01N 25/04; A01N 65/22; A01P 1/00

(57) ABSTRACT

The present disclosure provides an antimicrobial composition comprising micelles and a carrier. Each of the micelles comprises a core of one or more essential oils covered, at least in part, by one or more cationic surfactants. The antimicrobial compositions can be used with a liquid, gas, or solid carrier. Examples disclosed within include the use of a 1-octyl-3-methylimidazolium salt, a 1.3-didecyl-2-methylimidazolium salt, a 1-decyl-3-methylimidazolium salt, and combinations thereof with rosemary, thyme, mint, and eucalyptus oil.

### CROSS-REFERENCE TO RELATED APPLICATIONS

The present application is claiming priority from U.S. Provisional Application No. 63/167,298 filed Mar. 29, 2021, the content of which is hereby incorporated by reference in its entirety.

### TECHNOLOGICAL FIELD

The present invention pertains to antimicrobial compositions that reduce the viability of, inactivate or preventing the growth of bacteria, viruses and fungi. It also pertains to methods for preparing antimicrobial compositions and methods of using antimicrobial compositions to disinfect and/or sanitize a solid, liquid or gas.

### BACKGROUND

A wide range of pathogenic bacteria, viruses and fungi are capable of causing disease in humans and animals. Elimination of pathogenic microbes in household, healthcare, agricultural and industrial settings often relies on the use of disinfectants and sanitizing agents that contain toxic and irritating chemicals such as chlorine and its derivatives, alcohols, iodophors, ammonia, glutaraldehyde, ethylene oxide, phenols, formaldehyde and triclosan. While these disinfectants and sanitizing agents containing such chemicals may be effective at rapidly killing, inactivating or preventing the growth of pathogenic microbes, their composition can be acutely toxic, irritating, damaging and corrosive to materials and surfaces with which they come into contact. Moreover, long-term exposure to disinfectants and sanitizing agents that contain toxic and irritating chemicals has been associated with a myriad of adverse health effects including, but not limited to, the development of asthma, hormonal disruption and cancer. Additionally, as some of these harsh and toxic chemicals tend to be poorly biodegradable, they are also hazardous to the environment.

In recent years, greater attention has been given to the use of natural, non-toxic and environment-friendly/“green” alternatives to conventional disinfectants and sanitizing agents. However, such products are often unable to rapidly kill pathogenic bacteria and fungi, or are ineffective at inactivating viruses. Furthermore, despite being marketed as “natural” and/or “environmentally-safe” options, disinfecting and sanitizing products that are labeled as “green” may still contain toxic chemicals that are poorly biodegradable and hazardous to the environment.

Attempts have been made to formulate effective disinfectant and sanitizing solutions based on plant essential oils. Essential oils have long been recognized for their inherent antimicrobial properties. Some examples of essential oils with demonstrated antimicrobial activity include cinnamon oil, thyme oil, tea tree oil, oregano oil, peppermint oil, lemon oil and eucalyptus oil. These essential oils, and others, have been demonstrated to be effective at killing, inactivating or preventing the growth of a broad-range of pathogenic microbes including different bacteria (e.g. Streptococcus pyogenes, Streptococcus agalactiae, Streptococcus pneumoniae, Klebsiella pneumoniae, Staphylococcus aureus and Streptococcus maltophilia, etc.), fungi (e.g. Candida albicans, Aspergillus fumigatus, etc.) and viruses (e.g. influenza viruses, rhinoviruses, human herpes simplex viruses types 1 and 2, rotavirus, coronavirus, etc.). However, the use of essential oils in disinfectants and sanitizing agents is hampered by their hydrophobic nature, which renders them immiscible in water and other aqueous solutions. While emulsifying or dispersing agents, like surfactants, or organic solvent can be used to solubilize essential oils in aqueous solutions, in some cases, their presence may inhibit the antimicrobial activity of the essential oils. Moreover, some surfactants and organic solvents may themselves be toxic, irritating and/or harmful to the environment.

It would be highly desirable to be provided with effective antimicrobial compositions that can be used to kill, inactivate or prevent the growth of a broad range of microbial pathogens, in a variety of settings, which are non-toxic, non-irritating and readily biodegradable.

### BRIEF SUMMARY

The present disclosure concerns antimicrobial compositions comprising cationic surfactant(s) and essential oil(s) displaying potent, synergistic antimicrobial activity, whilst also remaining relatively non-toxic and readily biodegradable.

According to a first aspect, the present disclosure provides an antimicrobial composition comprising micelles and a carrier. Each of the micelles comprises a core of one or more essential oil covered, at least in part, by one or more cationic surfactant. In an embodiment, the one or more essential oil comprises at least one of an anise oil, an arose otto oil, a balsam oil, a barley oil, a basil oil, a bergamot oil, a chamomile oil, a cedar oil, a cinnamon oil, a citronella oil, a clove oil, a coriander oil, a cranberry oil, a cypress oil, an eucalyptus oil, an evening primrose oil, a fennel oil, a fir oil, a fleagrass oil, a frankincense oil, a geranium oil, a ginger oil, a grapefruit oil, a hops oil, a jasmine oil, a juniper oil, a lavender oil, a lemon oil, a lemongrass oil, a lime oil, a macadamia nut oil, a mandarin oil, a marjoram oil, a myrrh oil, a myrtle oil, a neroli oil, an ocmea origanum oil, an orange oil, an oregano oil, a patchouli oil, a pepper oil, a peppermint oil, a petitgrain oil, a pimento berries oil, a pine oil, a pulegium mint oil, a rose oil, a rosemary oil, a rosewood oil, a sage oil, a sandalwood oil, a sea fennel oil, a sesame oil, a spearmint oil, a spikenard oil, a tea tree oil, a thyme oil, a vervail oil, a vetiver oil, a violet oil, a wheat oil, a wintergreen oil or a combination thereof. In another embodiment, the one or more cationic surfactant comprises at least one of an ionic liquid. In still another embodiment, the at least one ionic liquid comprise an imidazolium-based surfactant, a benzimidazolium-based surfactant, a quaternary ammonium (QAC)-based surfactant, a pyridinium-based surfactant, a pyrrolidium-based surfactant, or a combination thereof. In yet another embodiment, the at least one ionic liquid comprises the imidazolium-based surfactant, the benzimidazolium-based surfactant, or a combination thereof. In an embodiment, the imidazolium-based surfactant comprises at least one of a 1-octyl-3-methylimidazolium salt, a 1,3-didecyl-2-methylimidazolium salt, a 1-decyl-3-methylimidazolium salt or a combination thereof.

In an embodiment, wherein the one or more essential oil is a Myrtus communis oil, Thymus capitatus oil, a Mentha pulegium oil, or a Eucalyptus cinerea oil.

In another embodiment, the benzimidazolium-based surfactant comprises a 1,12-Bis((1-methylbenzimidazolium)-3-yl)dodecane salt. In some embodiments, the imidazolium-based surfactant compound and/or the benzimidazolium-based surfactant comprises one or more cleavable ester, ether, amide or hydroxyl functional group present in a hydrophobic chain. In an embodiment, the molarity to volume ratio of cationic surfactant to the essential oil in the antimicrobial composition is equal to or less than 15:1.

In another embodiment, the antimicrobial composition comprises micelles have an average diameter of 1000 nm or less. In still another embodiment, the antimicrobial composition comprises micelles have an average diameter of 100 nm or less. In yet another embodiment, the antimicrobial composition comprises a first population of micelles having a first average diameter and a second population of micelles having a second average diameter, wherein the first average diameter is different from the second average diameter. In some embodiments, the carrier is an aqueous solution. In yet another embodiment, the carrier lacks an organic solvent. In some embodiments, the antimicrobial composition of is an emulsion. In a further embodiment, the carrier is a solid.

According to a second aspect, the present disclosure provides a method for making the antimicrobial composition described herein. The method comprises combining the one or more essential oil with the one or more cationic surfactant in an aqueous solution to obtain the micelles. In an embodiment, the diameter of the micelles is adjusted by changing the concentration of ionic liquids in the micelles.

According to a third aspect, the present disclosure provides a method of preventing microbial growth, reducing the viability of or inactivating microbes on a solid, in a liquid or in a gas. The method comprises contacting the antimicrobial composition described herein with the solid, the liquid or the gas. In an embodiment, the method provides a reduction of at least one log in the amount of the microbe is observed after contact with the antimicrobial composition. In an embodiment, the microbe is at least one of a bacteria, a virus and a fungi. In a specific embodiment, where the bacteria is Escherichia coli, Staphylococcus aureus, Salmonella spp., Listeria monocytogenes, Legionella pneumophila, Bacillus cereus, Streptococcus agalactiae, Staphylococcus epidermidis, Proteus vulgari, Klebsiella pneumoniae, Enterococcus faecalis, Listeria innocua, Pseudomonas putida, Clostridium perfringens, Shigella sonnei, Campylobacter jejuni or Pseudomonas aeruginosa. In another embodiment, the virus is influenza virus, herpes simplex virus 1, coronavirus, rhinovirus, or rotavirus. In a further embodiment, the fungi is Candida albicans, or Aspergillus fumigatus.

### BRIEF DESCRIPTION OF THE DRAWINGS

Reference will now be made to the accompanying drawings.

FIG. 1 provides log 10 of reduction of L. pneumophila suspensions at 108 CFU/ml exposed for 1 min vertexing with selected EO diluted in water.

FIG. 2 provides log 10 of reduction of S. aureus suspensions at 108 CFU/ml exposed for 1 min vertexing with selected EO in water.

FIG. 3 provides dynamic light scattering (DLS) measurements of aggregates (emulsions) freshly synthesized comprising IL-3 (A: 0.5 μM; B: 1 μM; and C: 2 μM) with menthe EO (D) in water. Menthe EO with 0.5 μM IL-3 revealed the average size of aggregates at 120 nm that increased to ˜4 μm for IL-3 at 2 μM.

FIG. 4 provides DLS measurements of aggregates freshly synthesized comprising IL-3 (A: 0.5 μM; B: 1 μM; and C: 2 μM) with Thyme EO (D) diluted in water. Thyme EO with 0.5 M IL-3 revealed the average size of aggregates at 650 nm that decreased to ˜120 nm for IL-3 at 2 μM. Thymol (major component of the thyme EO) is more aromatic and requires higher amount of IL-3 to better solubilize EO.

FIG. 5 provides an example of the structure of C27H45N402Br ionic liquid with amide interlink introduced to modify IL's biodegradability. Other interlinks, such as ester or ether could also be introduced for this purpose. The introduction of ester/amide bond in the hydrophobic chain of IL will provide the site for enzymatic hydrolysis which give imidazolium fragment along with primary alcohol that may be readily metabolized, hence increase the rate of biodegradation. The increase in the number of carbon (>C10) in the hydrophobic chain not only decreases the solubility of lonic liquids in water but also increases the toxicity to the tested species. Therefore, it is important to balance the length of the carbon chain, functional group, and counterion. We have designed a dipodal IL (having two hydrophobic carbon chains at positions 1 and 3 of the imidazolium ring), so that the maximum carbon in one chain should be less than 10.

### DETAILED DESCRIPTION

The present disclosure concerns the combination of one or more cationic surfactants with one or more essential oil to form micelles having antimicrobial properties (and in some embodiments, synergistic antimicrobial properties). The micelles can be dispersed in a liquid, gaseous or solid carrier, thereby forming antimicrobial compositions according to the present disclosure. In some embodiments, these antimicrobial compositions can be particularly advantageous because they can be biodegradable, biocompatible and/or non-toxic. In further embodiments, the biodegradability of the antimicrobial compositions can be modulated by selecting specific cationic surfactants. In some embodiments, each component of the antimicrobial composition is non-toxic and/or has an acceptable biodegradability rate. In additional embodiments, the cationic surfactants of the antimicrobial composition is biodegradable or can be modified to alter its biodegradability profiled (e.g., being biodegraded in more or less time).

### Antimicrobial Composition

The antimicrobial compositions of the present disclosure comprises at least one cationic surfactant which allows formulating the at least one essential oil-based emulsion. As known in the art, cationic surfactants are amphipathic molecules that comprise both positively charged groups (referred to as their “heads”) and at least one hydrophobic chain (referred to as their “tails”). It is also known that cationic surfactants generally exhibit broad spectrum antimicrobial activity against planktonic pathogens such a bacteria, enveloped viruses and fungi and, as such, are often used in disinfectants and cleaning products. It is believed that the antimicrobial activity of cationic surfactants stems from the ability of the positively charged heads to bind to and disrupt the negatively charged outer lipid membranes of above mentioned classes of microbes, resulting in increased membrane permeability and, eventually, lysis of the microbial cell or virus particle. Cationic surfactants that are inherently less toxic, less irritating and more biodegradable are particularly well-suited for use in antimicrobial compositions of the present disclosure.

In the context of the present disclosure, the antimicrobial composition can comprise a single type of cationic surfactant or a combination of more than one type of cationic surfactants. In some embodiments, the antimicrobial composition comprises 2, 3, 4, 5, 6, 7, 8, 9, 10 or more different types of cationic surfactants. In additional embodiments, the antimicrobial composition comprises a single type of cationic surfactants.

In an embodiment, the cationic surfactant comprises one or more ionic liquid. Ionic liquids are a class of purely ionic, salt-like organic materials that are liquid at unusually low temperatures, such as room temperature or even 0° C. In recent years, ionic liquid-based surfactants have drawn more and more attention because of their unique properties and superior aggregation behavior. In the context of the present disclosure, the ionic liquid includes a cation exhibiting antimicrobial properties. Examples of ionic liquid surfactants comprising a cation exhibiting antimicrobial properties include, without limitation, quaternary ammonium-based surfactants (e.g., surfactants having a quaternary ammonium cation), pyridinium-based surfactant (e.g., surfactants having a pyridinium cation), pyrrolidium-based surfactants (e.g., surfactants having a pyrrolidium cation), imidazolium-based surfactants (e.g., surfactants having a imidazolium cation) and benzimidazolium-based surfactants (e.g., surfactants having a benzimidazolium cation). In some embodiments, the counter-anion of these cationic surfactants can be selected in function of the size of the micelle and/or the biodegradability properties that is being sought. In some embodiments, the molecular weight of the counter-anion is substantially smaller than the molecular weight of the cationic “head” of the surfactant. The counter-anion of these cationic surfactants can be, without limitation, a chloride, a bromide anion, an iodide anion, an acetate anion, an amino-acetate anion, an amino-propionate anion, a lactate anion or a methyl carbonate anion.

In the context of the present disclosure, the antimicrobial composition can comprise only a single type of ionic liquid surfactant or a combination of more than one type of ionic liquid surfactants. In some embodiments, the antimicrobial composition comprises 2, 3, 4, 5, 6, 7, 8, 9, 10 or more different types of ionic liquid surfactants. In additional embodiments, the antimicrobial composition comprises a single type of ionic liquid surfactant.

Exemplary quaternary ammonium-based surfactants that have been previously used as disinfectants and antimicrobial agents include, for example, a benzalkonium salt, a benzethonium salt, a methylbenzethonium salt, a cetrimonium salt, a dofanium salt, a tetraethylammonium salt, a didecdimethylammonium salt as well as combinations thereof.

Pyridinium cations are positively charged molecular ions derived from pyridine.

Pyrrolidinum cations are positively charged molecular ions derived from pyrrole.

Without wishing to be bound to theory, the planar imidazolium and benzimidazolium rings stabilize the positive charge due to delocalization of the IT electron. This represents a class of cationic surfactants attractive for their antibacterial activity.

Imidazolium cations are positively charged molecular ions derived from imidazole. Exemplary imidazolium-based surfactant include, but are not limited to a 1-octyl-3-methylimidazolium salt, a 1,3-didecyl-2-methylimidazolium salt, a 1-decyl-3-methylimidazolium salt, or a combination thereof. In some further embodiments, the imidazolium-based surfactant can include, without limitation, 1-Allyl-3-methylimidazolium bis(trifluoromethylsulfonyl)imide, 1-Allyl-3-methylimidazolium bromide, 1-Allyl-3-methylimidazolium chloride, 1-Allyl-3-methylimidazolium dicyanamide, 1-Allyl-3-methylimidazolium iodide, 1-Benzyl-3-methylimidazolium chloride, 1-Benzyl-3-methylimidazolium tetrafluoroborate, 1,3-Bis(cyanomethyl)imidazolium chloride, 1-Butyl-2,3-dimethylimidazolium chloride, 1-Butyl-2,3-dimethylimidazolium hexafluorophosphate, 1-Butyl-2,3-dimethylimidazolium tetrafluoroborate, 1-Butyl-2,3-dimethylimidazolium tetrafluoroborate, 4-(3-Butyl-1-imidazolio)-1-butanesulfonate, 1-Butyl-3-methylimidazolium acetate, 1-Butyl-3-methylimidazolium acetate, 1-Butyl-3-methylimidazolium bis(trifluoromethylsulfonyl)imide, 1-Butyl-3-methylimidazolium bis(trifluoromethylsulfonyl)imide, 1-Butyl-3-methylimidazolium bis(trifluoromethylsulfonyl)imide Solarpur®, 1-Butyl-3-methylimidazolium bromide, 1-Butyl-3-methylimidazolium bromide, 1-Butyl-3-methylimidazolium chloride, 1-Butyl-3-methylimidazolium chloride, 1-Butyl-3-methylimidazolium dicyanamide, 1-Butyl-3-methylimidazolium hexafluoroantimonate, 1-Butyl-3-methylimidazolium hexafluorophosphate, 1-Butyl-3-methylimidazolium hexafluorophosphate, 1-Butyl-3-methylimidazolium hydrogen sulfate, 1-Butyl-3-methylimidazolium iodide, 1-Butyl-3-methylimidazolium methanesulfonate, 1-Butyl-3-methylimidazolium methyl sulfate, 1-Butyl-3-methylimidazolium methyl sulfate, 1-Butyl-3-methylimidazolium nitrate, 1-Butyl-3-methylimidazolium octyl sulfate, 1-Butyl-3-methylimidazolium tetrachloroaluminate, 1-Butyl-3-methylimidazolium tetrafluoroborate, 1-Butyl-3-methylimidazolium tetrafluoroborate, 1-Butyl-3-methylimidazolium tetrafluoroborate, 1-Butyl-3-methylimidazolium thiocyanate, 1-Butyl-3-methylimidazolium thiocyanate, 1-Butyl-3-methylimidazolium trifluoromethanesulfonate, 1-Butyl-3-methylimidazolium trifluoromethanesulfonate, 1-(3-Cyanopropyl)-3-methylimidazolium bis(trifluoromethylsulfonyl)amide, 1-(3-Cyanopropyl)-3-methylimidazolium chloride, 1-(3-Cyanopropyl)-3-methylimidazolium dicyanamide, 1-Decyl-3-methylimidazolium chloride, 1-Decyl-3-methylimidazolium tetrafluoroborate, 1,3-Diethoxyimidazolium bis(trifluoromethylsulfonyl)imide, 1,3-Diethoxyimidazolium hexafluorophosphate, 1,3-Dihydroxyimidazolium bis(trifluoromethylsulfonyl)imide, 1,3-Dihydroxy-2-methylimidazolium bis(trifluoromethylsulfonyl)imide, 1,3-Dimethoxyimidazolium bis(trifluoromethyl-sulfonyl)imide, 1,3-Dimethoxyimidazolium hexafluorophosphate, 1,3-Dimethoxy-2-methylimidazolium bis(trifluoromethylsulfonyl)imide, 1,3-Dimethoxy-2-methylimidazolium hexafluorophosphate, 1,3-Dimethylimidazolium dimethyl phosphate, 1,3-Dimethylimidazolium methyl sulfate, 1,2-Dimethyl-3-propylimidazolium bis (trifluoromethylsulfonyl)imide, 1,2-Dimethyl-3-propylimidazolium tris(trifluoromethylsulfonyl)methide, 1-Dodecyl-3-methylimidazolium iodide, 1-Ethyl-2,3-dimethylimidazolium tetrafluoroborate, 1-Ethyl-3-methylimidazolium acetate, 1-Ethyl-3-methylimidazolium acetate, 1-Ethyl-3-methylimidazolium aminoacetate, 1-Ethyl-3-methylimidazolium (S)-2-aminopropionate, 1-Ethyl-3-methylimidazolium bis(pentafluoroethylsulfonyl)imide, 1-Ethyl-3-methylimidazolium bis(trifluoromethylsulfonyl)imide, 1-Ethyl-3-methylimidazolium bis(trifluoromethylsulfonyl)imide, 1-Ethyl-3-methylimidazolium bromide, 1-Ethyl-3-methylimidazolium bromide, 1-Ethyl-3-methylimidazolium chloride, 1-Ethyl-3-methylimidazolium chloride, 1-Ethyl-3-methylimidazolium chloride, 1-Ethyl-3-methylimidazolium chloride-aluminum chloride, 1-Ethyl-3-methylimidazolium dicyanamide, 1-Ethyl-3-methylimidazolium dicyanamide, 1-Ethyl-3-methylimidazolium diethyl phosphate, 1-Ethyl-3-methylimidazolium diethyl phosphate, 1-Ethyl-3-methylimidazolium dimethyl phosphate, 1-Ethyl-3-methylimidazolium ethyl sulfate, 1-Ethyl-3-methylimidazolium ethyl sulfate, 1-Ethyl-3-methylimidazolium hexafluorophosphate, 1-Ethyl-3-methylimidazolium hexafluorophosphate, 1-Ethyl-3-methylimidazolium hydrogen sulfate, 1-Ethyl-3-methylimidazolium iodide, 1-Ethyl-3-methylimidazolium L-(+)-lactate, 1-Ethyl-3-methylimidazolium methanesulfonate, 1-Ethyl-3-methylimidazolium methyl sulfate, 1-Ethyl-3-methylimidazolium tetrachloroaluminate, 1-Ethyl-3-methylimidazolium tetrachloroaluminate, 1-Ethyl-3-methylimidazolium tetrafluoroborate, 1-Ethyl-3-methylimidazolium tetrafluoroborate, 1-Ethyl-3-methylimidazolium tetrafluoroborate, 1-Ethyl-3-methylimidazolium 1,1,2,2-tetrafluoroethanesulfonate, 1-Ethyl-3-methylimidazolium thiocyanate, 1-Ethyl-3-methylimidazolium tosylate, 1-Ethyl-3-methylimidazolium trifluoromethanesulfonate, 1-Ethyl-3-methylimidazolium trifluoromethanesulfonate, 1-Ethyl-3-methylimidazolium trifluoromethanesulfonate, 1-Hexyl-3-methylimidazolium bis(trifluormethylsulfonyl)imide, 1-Hexyl-3-methylimidazolium chloride, 1-Hexyl-3-methylimidazolium chloride, 1-Hexyl-3-methylimidazolium hexafluorophosphate, 1-Hexyl-3-methylimidazolium iodide, 1-Hexyl-3-methylimidazolium tetrafluoroborate, 1-(2-Hydroxyethyl)-3-methylimidazolium dicyanamide, 1-Methylimidazolium chloride, 1-Methylimidazolium hydrogen sulfate, 1-Methyl-3-octylimidazolium chloride, 1-Methyl-3-octylimidazolium hexafluorophosphate, 1-Methyl-3-octylimidazolium tetrafluoroborate, 1-Methyl-3-propylimidazolium iodide, 1-Methyl-3-propylimidazolium methyl carbonate, 1-Propyl-2,3-dimethyl-imidazolium iodide Solarpur®, 1-Propyl-3-methylimidazolium bis(trifluoromethylsulfonyl)imide, 1-Propyl-3-methylimidazolium bis(trifluoromethylsulfonyl)imide Solarpur® or combinations thereof.

In some embodiments, the 1-octyl-3-methylimidazolium salt is 1-octyl-3-methylimidazolium chloride. In additional embodiments, the 1,3-didecyl-2-methylimidazolium salt is 1,3-didecyl-2-methylimidazolium chloride. In yet additional embodiments, the 1-decyl-3-methylimidazolium-based salt is 1-decyl-3-methylimidazolium chloride.

In yet an additional embodiment, the antimicrobial composition comprises one or more imidazolium-based surfactant. In some embodiments, the antimicrobial composition comprises 2, 3, 4, 5, 6, 7, 8, 9, 10 or more different types of imidazolium-based surfactants. For example, the antimicrobial composition can include a single imidazolium-based surfactant.

Benzimidazolium cations are positively charged molecular ions derived from benzimidazole, a bicyclic compound that consists of a fusion of benzene and imidazole. Exemplary benzimidazolium-based surfactant include, but are not limited to a 1,12-Bis((1-methylbenzimidazolium)-3-yl)dodecane. In some embodiments, the 1,12-Bis((1-methylbenzimidazolium)-3-yl)dodecane salt is a 1,12-Bis((1-methylbenzimidazolium)-3-yl)dodecane bromide.

In yet an additional embodiment, the antimicrobial composition comprises one or more benzimidazolium-based surfactant. In some embodiments, the antimicrobial composition comprises 2, 3, 4, 5, 6, 7, 8, 9, 10 or more different types of benzimidazolium-based surfactants. For example, the antimicrobial composition can include a single benzimidazolium-based surfactant. In yet additional embodiments, the antimicrobial composition can include both one or more imidazolium-based surfactant and one or more benzimidazolium-based surfactant.

As indicated above, in some embodiments, the ionic liquid surfactant comprises an imidazolium-based surfactant, a benzimidazolium-based surfactant or a combination thereof. These particular surfactants include delocalized TT (pi) electrons in their imidazole ring which increases their thermal and chemical stability. Consequently, the stability and ultimately the biodegradability of these surfactants can be modulated by including one or more cleavable functional group in a hydrophobic chain within the molecule (e.g. an alkyl chain). Such modification can make them biodegrade more rapidly than their counterpart lacking such modification. The cleavable functional groups that can be included in the hydrophobic chain of the imidazolium-based surfactant and/or the benzimidazolium-based surfactant can be, without limitation, an ester group, an ether group, an amide group or a hydroxyl group. Once incorporated into the hydrophobic chain of the ionic liquid surfactant, cleavage of said functional groups will cleave said alkyl hydrophobic into two more parts, thereby increasing the surfactants' biodegradability rate. Alternatively or in combination, it is also possible to adjust (e.g., increase or decrease) the length of the hydrophobic chain of these ionic liquid surfactants to modulate (e.g., reduce) their biodegradability rates while maintaining strong antibacterial activity and minimized cytotoxicity. In some embodiments, the modified imidazolium-based surfactant and/or benzimidazolium-based surfactant can be biodegraded within 1 month, 1 week, 6 days, 5 days, 4 days, 3 days, 2 days or less. In additional embodiments, the antimicrobial composition can comprises more than one type of modified imidazolium-based surfactant and/or benzimidazolium-based surfactant, each type of surfactant having a distinct biodegradability rate to provide an antimicrobial composition having surfactants with a range of biodegradability rates.

The antimicrobial composition of the present disclosure includes one or more essential oil that is formulated with the cationic surfactant. The essential oil or mixtures of essential oils present in the antimicrobial composition exhibit antimicrobial properties. As it is known in the art, essential oils comprise complex volatile compounds, synthesized naturally in different plant parts during the process of secondary metabolism present in an oil obtained from a plant. The antimicrobial properties of essential oils and other plant extracts have been recognized for many years. In particular, it is known that essential oils comprise different chemical constituents, such as aldehydes, phenolics, terpenes and other compounds, that possess antimicrobial activity against bacteria, viruses and fungi.

The essential oil used in the antimicrobial composition of the present invention may be any one of an anise oil, an arose otto oil, a balsam oil, a barley oil, a basil oil, a bergamot oil, a chamomile oil, a cedar oil, a cinnamon oil, a citronella oil, a clove oil, a coriander oil, a cranberry oil, a cypress oil, an eucalyptus oil, an evening primrose oil, a fennel oil, a fir oil, a fleagrass oil, a frankincense oil, a geranium oil, a ginger oil, a grapefruit oil, a hops oil, a jasmine oil, a juniper oil, a lavender oil, a lemon oil, a lemongrass oil, a lime oil, a macadamia nut oil, a mandarin oil, a marjoram oil, a myrrh oil, a myrtle oil, a neroli oil, an ocmea origanum oil, an orange oil, an oregano oil, a patchouli oil, a pepper oil, a peppermint oil, a petitgrain oil, a pimento berries oil, a pine oil, a pulegium mint oil, a rose oil, a rosemary oil, a rosewood oil, a sage oil, a sandalwood oil, a sea fennel oil, a sesame oil, a spearmint oil, a spikenard oil, a tea tree oil, a thyme oil, a vervail oil, a vetiver oil, a violet oil, a wheat oil, a wintergreen oil or a combination thereof. The essential oil used in the antimicrobial composition of the present disclosure can be a single type of essential oil or a combination of different essential oils. In some embodiments, at least 2, 3, 4, 5, 6, 7, 8, 9 or 10 different types of essential oils are included in the antimicrobial composition. In additional embodiments, a single type of essential oil is present in the antimicrobial composition. In a specific example, a rosemary oil is present in the antimicrobial composition of the present disclosure. The essential oil may be of natural origin, synthetic origin, or a combination thereof.

The composition of the present disclosure is considered “antimicrobial” because it exhibits, in some embodiments, the ability to reduce the viability of a microbe (microbicide) and/or prevent microbial growth or replication (microbiostatic). The composition of the present disclosure can exhibit antimicrobial activity against any microbes such as bacteria (e.g. Escherichia coli, Staphylococcus aureus, Salmonella spp., Listeria monocytogenes, Legionella pneumophila, Bacillus cereus, Streptococcus agalactiae, Staphylococcus epidermidis, Proteus vulgari, Klebsiella pneumoniae, Enterococcus faecalis, Listeria innocua, Pseudomonas putida, Clostridium perfringens, Shigella sonnei, Campylobacter jejuni and/or Pseudomonas aeruginosa), viruses (e.g. influenza virus, herpes simplex virus 1, coronavirus, rhinovirus, rotavirus, etc.) and/or fungi (e.g. Candida albicans, Aspergillus fumigatus, etc.). In an embodiment, the antimicrobial composition exhibits antibacterial activity. In another embodiment, the antimicrobial composition exhibits antiviral activity. In some embodiments, treatment of a population of microbes with the antimicrobial composition of the present disclosure decreases the growth, replication or viability by at least 1, 2, 3, 4, 5 or 6 logs within 1, 2, 3, 4 or 5 minutes compared to a control composition lacking antimicrobial activity.

The antimicrobial composition of the present disclosure includes micelles which comprises both the cationic surfactant and the essential oil. The term “micelles” as used herein refers to stable spherical or near-spherical aggregates comprising both the essential oil and the cationic surfactant. The micelles have a core comprising at least one essential oil which is covered, at least in part, by one or more cationic surfactants. In the context of the present disclosure, the expression “covered at least in part” refers to the fact that at least 1, 2, 5, 10, 15, 20, 25, 30, 35, 40, 45, 50, 55, 60, 65, 70, 75, 80, 85, 90, 95, 96, 97, 98, 99% of the core of the micelle is covered with at least one cationic surfactant. In some embodiments, the micelles are covered entirely with a layer formed by at least one cationic surfactant. In the micelles, where the positively charged heads of the surfactant are in contact with the external milieu (in some embodiments, an aqueous solution) and the hydrophobic tails is oriented towards the core of the aggregate (formed by the one or more essential oils).

The micelles present in the antimicrobial compositions of the invention may be uniform in size or may comprise different sizes. In some embodiments, the antimicrobial compositions comprises different population of micelles, each population of micelles having a distinct average diameter. In some embodiments, micelles present in the antimicrobial composition of the present disclosure may have average diameters equal to or less than 1000 nm, or equal to or less than 950 nm, or equal to or less than 900 nm, or equal to or less than 850 nm, or equal to or less than 800 nm or equal to or less than 750 nm, or equal to or less than 700 nm, or equal to or less than 650 nm, or equal to or less than 600 nm, or equal to or less than 550 nm, or equal to or less than 500 nm, or equal to or less than 450 nm, or equal to or less than 400 nm, or equal to or less than 350 nm, or equal to or less than 300 nm, or equal to or less than 250 nm, or equal to or less than 200 nm, or equal or less than 150 nm, or equal or less than 100 nm, or equal or less than 50 nm, or equal or less than 40 nm, or equal or less than 30 nm, or equal or less than 20 nm, or equal or less than 10 nm. In some embodiments, micelles present in the antimicrobial composition of the present disclosure may have average diameters equal to or higher than 1 nm, equal to or higher than 2 nm, equal to or higher than 3 nm, equal to or higher than 4 nm, equal to or higher than 5 nm, equal to or higher than 6 nm, equal to or higher than 7 nm, equal to or higher than 8 nm, equal to or higher than 9 nm, equal to or higher than 10 nm, equal to or higher than 15 nm, equal to or higher than 20 nm, equal to or higher than 25 nm, equal to or higher than 30 nm, equal to or higher than 35 nm, equal to or higher than 40 nm, equal to or higher than 45 nm, equal to or higher than 50 nm, equal to or higher than 55 nm, equal to or higher than 60 nm, equal to or higher than 65 nm, equal to or higher than 70 nm, equal to or higher than 75 nm, equal to or higher than 80 nm, equal to or higher than 85 nm, equal to or higher than 90 nm, equal to or higher than 100 nm, equal to or higher than 150 nm, equal to or higher than 200 nm, equal to or higher than 250 nm, equal to or higher than 300 nm, equal to or higher than 350 nm, equal to or higher than 400 nm, equal to or higher than 450 nm, equal to or higher than 500 nm, equal to or higher than 550 nm, equal to or higher than 600 nm, equal to or higher than 650 nm, equal to or higher than 700 nm, equal to or higher than 750 nm, equal to or higher than 800 nm, equal to or higher than 850 nm, equal to or higher than 900 nm, equal to or higher than 950 nm or equal to or higher than 1000 nm. In some specific embodiments, the micelles have an average diameter between 500 and 1500 nm, for example 1000 nm or less. In some embodiments, when the antimicrobial composition is intended to exhibit antibacterial activity, the micelles can have an average diameter equal to or below 2000 or 1000 nm. In some additional embodiments, the micelles have an average diameter between 50 and 150 nm, for example, 100 nm or less. In some embodiments, when the antimicrobial composition is intended to exhibit antiviral, the micelles can have an average diameter equal to or below 100 nm.

In an embodiment, the micelles can be comparable or smaller than dimensions of typical viral particles.

In the antimicrobial composition, the micelles are provided with a carrier. The carrier is in physical contact with the micelles. The term “carrier” described herein refers to a liquid, gas or solid within which the antimicrobial micelles may be applied to, suspended, dispersed, solubilized or emulsified. Examples of suitable liquid carriers include, water or other aqueous solutions (which may include, for example, a buffering agent). In additional embodiments, when the carrier is a liquid, the antimicrobial composition can be an emulsion. The term “emulsion” described herein refers to a colloidal suspension of micelles, as described above, dispersed within an aqueous solution. In embodiments, the aqueous solution lacks an organic solvent. Examples of suitable gaseous carriers include, for example, air, nitrogen, oxide and carbon dioxide. In additional embodiments, when the carrier is a gas, the micelles can be aerosolized in the gas. The term “aerosolized” described herein refers to the process of converting the antimicrobial composition of the present disclosure into a suspension of particles or liquid droplets that are small and light enough to be carried in the air or another gas. Examples of suitable solid carriers include, for example, textiles, wood, wood derivatives, plastics, cellulose, starch, cyclodextrin, silicates, silica gels and waxes. In some additional embodiments, when the carrier is a solid, the antimicrobial composition can be provided on the solid carrier as a liquid or a gas form.

In some embodiments, the cationic surfactant is provided at a certain molarity to volume ratio with respect to the essential oil. The term “molarity to volume ratio” described herein refers to the proportion of cationic surfactant in an antimicrobial composition, measured by its total molar concentration, relative to the proportion of essential oil, measured by its total volume. The molarity (μM) to volume (μL) ratio of surfactant to essential oil in antimicrobial compositions of this invention may be equal to or less than 15:1, or equal to or less than 14:1, or equal to or less than 13:1, or equal to or less than 12:1, or equal to or less than 11:1, or equal to or less than 10:1, or equal to or less than 9:1, or equal to or less than 8:1, or equal to or less than 7:1, or equal to or less than 6:1, or equal to or less than 5:1, or equal to or less than 4:1, or equal to or less than 3:1, or equal to or less than 2:1, or equal to or less than 1:1.

### Method for Making the Antimicrobial Composition

A method of making the antimicrobial compositions of the present disclosure comprises combining one or more essential oil with one or more cationic surfactant in an aqueous solution in order to obtain micelles. The term “combining” described herein refers to the stepwise admixing of the essential oil and the cationic surfactant. The micelles can be formed by combining the at least one surfactant and the at least one essential oil in an aqueous solution. The method can include submitting the combination of essential oil/cationic surfactant to agitation to provide the micelles. In some embodiments, the method lack including an organic solvent to the aqueous solution or to the composition (prior to or after the combining step). The antimicrobial composition of the present disclosure can be made and used without any additional steps. However, in additional embodiments, the antimicrobial composition can be made in a concentrated form and the method of making the antimicrobial compositions can further comprise the step of diluting the composition (prior to use). Diluents such as water or other aqueous solutions, which may or may not be identical to the aqueous solution used to obtain micelles, may be employed. In additional embodiments, the antimicrobial composition can be made in a diluted form and the method of making the antimicrobial compositions can further comprise the step of concentrating the composition (prior to use). Concentration of the antimicrobial composition may be carried out by removing and/or evaporating some or all of the aqueous solution using standard procedures known in the art. In some additional embodiments, the antimicrobial composition can be dried to provide the micelles in a dried form. For example, the antimicrobial composition can be dried on a solid carrier (such as, for example, on a bead or a textile). In yet additional embodiments, the method of making the antimicrobial compositions further comprises the step of aerosolizing the composition in gaseous carriers such as air, nitrogen, oxide and carbon dioxide. Aerosolization of the micelles may be carried using standard procedures known in the art.

### Use of the Antimicrobial Composition

The antimicrobial composition of the present disclosure can be used to reduce the viability of or, in some embodiments, kill a microbe on a solid, in a liquid or in a gas. In order to do so, the antimicrobial composition is contacted the solid, liquid or gas wherein the microbe is found. The term “contacting” described herein refers to putting the antimicrobial composition in physical contact with the solid, liquid or gas, and comprises, for example, spraying, pouring, spreading, coating, rubbing or dusting the solid, liquid or gas with the antimicrobial composition. In additional embodiments, the antimicrobial composition can lead to a reduction of at least one log (and is some embodiments, at les 2, 3, 4, 5, 6 or more log) in the amount of microbes observed on the solid, in the liquid or in the gas after contact with the antimicrobial composition (when compared to a control composition lacking antimicrobial activity tested under similar conditions). The term “a reduction of at least one log” described herein refers to inactivating or eliminating 90, 91, 92, 93, 94, 95, 96, 97, 98, 99% or more of the microbes in the microbial population targeted. This reduction in the amount of microbes can be observed rapidly, in less than 5, 4, 3, 2, 1 minutes or less. This reduction in the amount of microbes can be observed when the contacting step occurs at room temperature (e.g., between 20 and 30° C.).

The antimicrobial compositions of the present disclosure to prevent microbial growth on a solid, in a liquid or in a gas comprises contacting the composition with the solid, liquid or gas. The term “prevent microbial growth” described herein refers to preventing the attachment, colonization and/or growth of microbes on the solid, in the liquid or in the gas. The term “prevent microbial growth” described herein refers to a reduction in the presence of microbes on the solid, liquid or gas when compared to a control composition lacking antimicrobial activity (tested under similar conditions).

The antimicrobial composition can be provided in a container and optionally in combination with instructions on how to use the composition. The antimicrobial composition can be provided on a solid surface, a textile for example.

### EXAMPLE

To prepare the micelles, a standard solution of ILs were prepared at concentration of 1 mM. Different compositions of ionic liquids (ILs) and essential oil (EOs) were mixed together and vortexed for 2 minutes (as set forth in Table 1) and the size of the micelles obtained was determined using dynamic light scattering. The ionic liquids self-assembled in such a way that hydrophilic imidazolium group interacts with water and stay outside, whereas hydrophobic unit interacts with essential oil and occupies core of spherical micelles. The diameter of the compositions made was assessed using dynamic light scattering and these results are shown in Table 1.

The ionic liquids investigated individually or in compositions with EOs are the following:

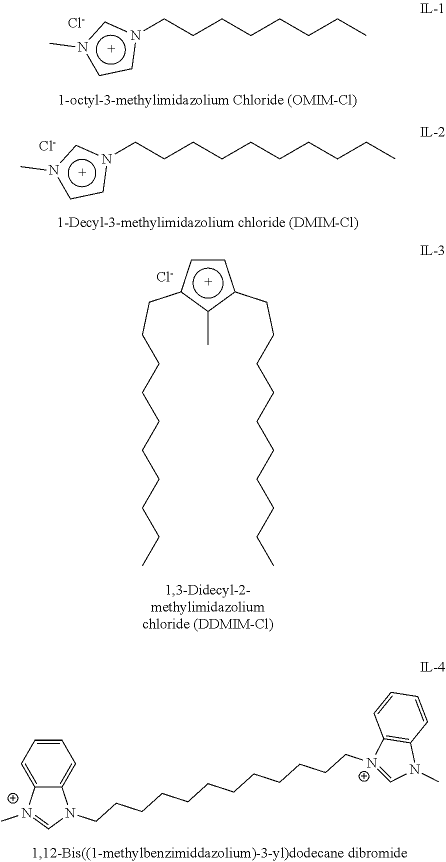

TABLE 1
Compositions and diameters of selected micelles. Compositions #8 and
9 concern a rosemary oil sourced from Tunisia (TN RO). The other
compositions contain commercial rosemary oil (com RO).

| # | Surfactant | Rosemary oil | Relative diameter of the micelles | |
|---|---|---|---|---|
| 1 | 1 mM IL-1 | — | 1000 | nm |
| 2 | 100 μM IL-1 | — | No micelle obtained | |
| 3 | 80 μM IL-1 | 7.5 μL | 1100 | nm |
| 4 | 1 mM IL-2 | — | 1000 | nm |
| 5 | 100 μM IL-2 | — | No micelle obtained | |
| 6 | 10 μM IL-2 | 7.5 μL | N.D. | |
| 7 | 100 μM IL-3 | 7.5 μL | 300-400 | nm |
| 8 | 5 μM IL-3 | 2.5 μL | 120 | nm* |

| | | | |
|---|---|---|---|
| 9 | 5 μM IL-3 | 2.5 μL | ~175 nm (after |
| | | | 3 weeks) |

*Determined upon a 10X dilution

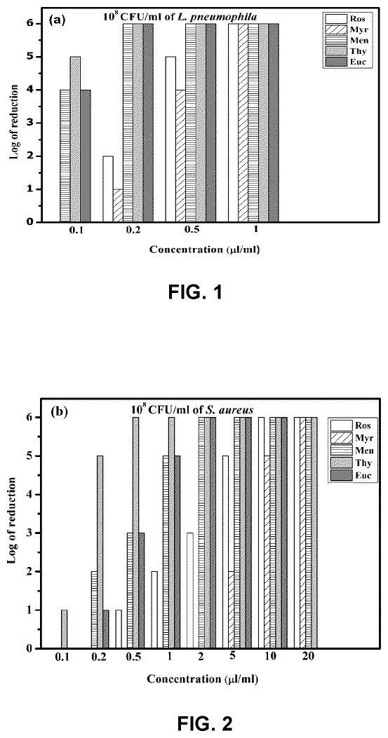

Composition #9 corresponds to composition #8 that was stored for 3 weeks at room temperature and the relative diameter of its micelles was determined using dynamic light scattering (upon a 10X dilution). As shown on FIGS. 1 and 2, no significant change in the diameter of the micelles was observed during storage. The size distribution again lied in the range of 100-200 nm.

The antimicrobial activity of various compositions was characterized. Bacterial suspensions at 108 CFU/ml were exposed to individual EOs and ILs, and to ILs-EO compositions at different concentrations. Commercial and Tunisian RO (com RO and TN RO), Tunisian Myrtus communis (TN Myr), Thymus capitatus (TN Thym), Mentha pulegium (TN Men), and Eucalyptus cinerea (TN Euc) were investigated. The bactericidal properties were obtained for the exposure time of 1 min. Following the exposure, 100 μl of each sample was dispensed in agar (LB for E. coli and S. aureus, and BCYE for L. pneumophila). The log 10 reduction (from 0 to 6) was then calculated by counting the number of colonies grown from samples treated with different solutions versus the number of colonies grown from 100 μl of non-treated bacterial suspensions. The results obtained are presented in Tables 2 to 5.

TABLE 2
Bactericidal properties of compounds comprising a rosemary
essential oil (com RO). Results are shown as log₁₀ reduction
when compared to an untreated sample.

| Formulation | | |
|---|---|---|
| in 1 ml water | E. coli | L. pneumophila |
| RO 2.5 μl | 0 | 0 |
| RO 5 μl | 4 | 3 |
| RO 7.5 μl | 5 | 4 |
| RO 10 μl | 6 | 6 |

TABLE 3
Bactericidal properties of IL-1 and the compound
of IL-1 with com RO. . Results are shown as log₁₀ reduction
when compared to an untreated sample.

| Formulation | | |
|---|---|---|
| in 1 ml water | E. coli | L. pneumophila |
| 30 mM IL-1 | 0 | |
| 40 mM IL-1 | 4 | |
| 60 mM IL-1 | 4-5 | |
| 80 mM IL-1 | 5 | 4 |
| 100 mM IL-1 | 6 | 6 |
| 80 mM IL-1 + 7.5 μl RO | 6 | 6 |

TABLE 4
Bactericidal properties of compounds comprising a IL-2
and compounds of IL-2 with com RO. Results are shown as
log₁₀ reduction when compared to an untreated sample.

| Formulation | | |
|---|---|---|
| in 1 ml water | E. coli | L. pneumophila |
| 1 mM IL-2 | 5 | 4 |
| 10 mM IL-2 | 6 | 6 |
| 20 mM IL-2 | 6 | |
| 10 μM IL-2 + 7.5 μl RO | 4 | 6 |
| 100 μM IL-2 + 7.5 μl RO | 5 | 6 |
| 1 mM IL-2 + 7.5 μl RO | 6 | |

TABLE 5
Bactericidal properties of IL-3 and compounds
of IL-3 with com RO, TN RO, TN Myr, TN Men, TN
Euc and TN Thym. Results are shown as log₁₀ reduction
when compared to an untreated sample.

| Formulation | | | |
|---|---|---|---|
| in 1 mL water | E. coli | L. pneumophila | S. aureus |
| 5 μM IL-3 | | | 4 |
| 10 μM IL-3 | | 0 | 6 |
| 50 μM IL-3 | | 4 | 6 |
| 100 μM IL-3 | 5 | 6 | |
| 1 mM IL-3 | 6 | 6 | |
| 10 mM IL-3 | 6 | | |
| 20 mM IL-3 | 6 | | |
| 10 μM IL-3 + 7.5 μl com RO | 6 | 6 | |
| 50 μM IL-3 + 7.5 μl com RO | 6 | 6 | |
| 100 μM IL-3 + 7.5 μl com RO | 6 | | |
| 1 mM IL-3 + 7.5 μl com RO | 6 | | |
| 10 mM IL-3 + 7.5 μl com RO | 6 | | |
| 20 mM IL-3 + 7.5 μl com RO | 6 | | |
| 2.5 μM IL-3 + 0.5 μl TN RO | | 6 | 6 |
| 2.5 μM IL-3 + 1 μl TN RO | | 6 | 6 |
| 2.5 μM IL-3 + 0.5 μl TN Myr | | 6 | |
| 2.5 μM IL-3 + 0.1 μl TN Men | | 6 | |
| 2.5 μM IL-3 + 0.2 μl TN Men | | | 5 |
| 2.5 μM IL-3 + 0.5 μl TN Men | | | 6 |
| 2.5 μM IL-3 + 0.5 μl TN Euc | | | 6 |
| 2.5 μM IL-3 + 0.1 μl TN Thym | | | 6 |

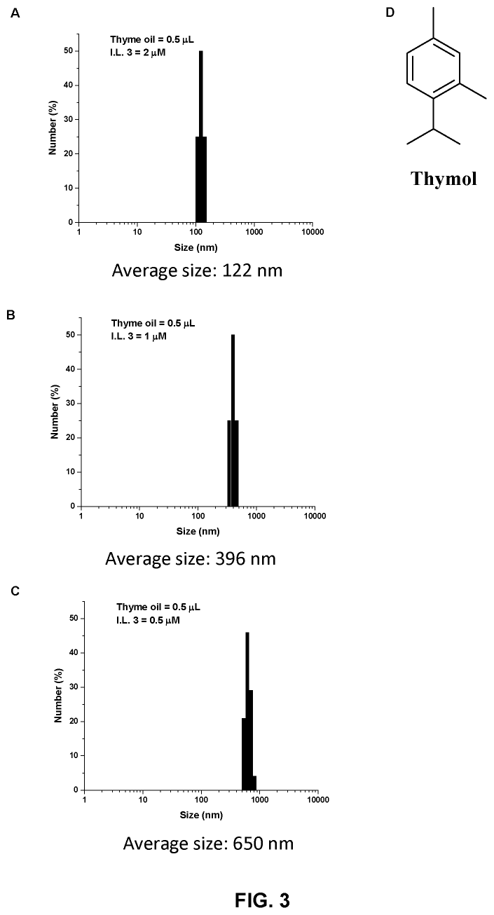

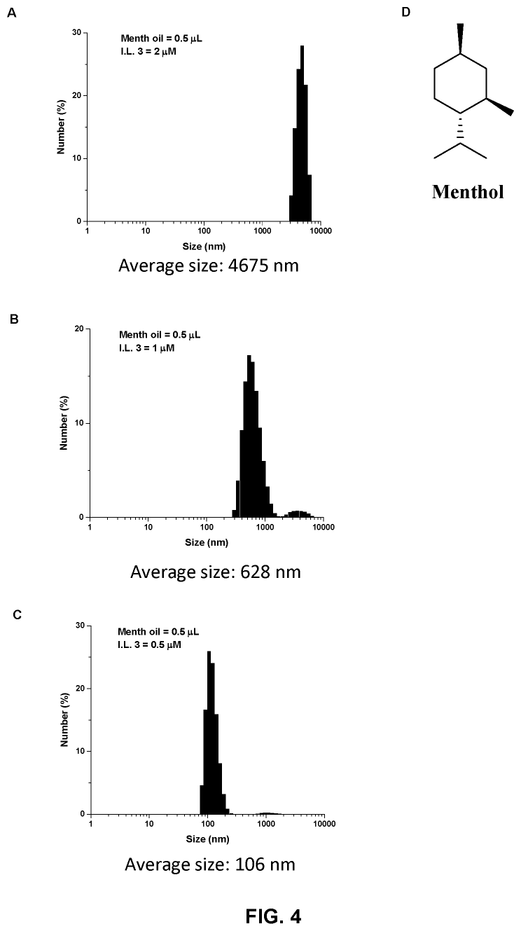

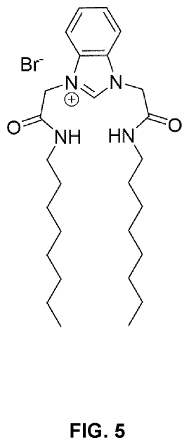

Examples of menthe and thyme oil micelles formed with IL-3 and diluted in water are shown in FIGS. 3 and 4, respectively. Due to different mechanisms of EO-IL interaction and molecular difference in composition of ingredients, the diameter of menthe-based micelles increases with the increasing concentration of IL, while the opposite effect is observed for thyme-based micelles. Of particular interest to the efficient interaction with bacteria is formation of ˜100 nm micelles as reported in FIGS. 3 and 4.

While the invention has been described in connection with specific embodiments thereof, it will be understood that the scope of the claims should not be limited by the preferred embodiments set forth in the examples, but should be given the broadest interpretation consistent with the description as a whole.

1. An antimicrobial composition comprising micelles and a carrier, wherein each of the micelles comprises a core of one or more essential oil covered, at least in part, by one or more cationic surfactant.

2. The antimicrobial composition of claim 1, wherein the one or more essential oil comprises at least one of an anise oil, an arose otto oil, a balsam oil, a barley oil, a basil oil, a bergamot oil, a chamomile oil, a cedar oil, a cinnamon oil, a citronella oil, a clove oil, a coriander oil, a cranberry oil, a cypress oil, an eucalyptus oil, an evening primrose oil, a fennel oil, a fir oil, a fleagrass oil, a frankincense oil, a geranium oil, a ginger oil, a grapefruit oil, a hops oil, a jasmine oil, a juniper oil, a lavender oil, a lemon oil, a lemongrass oil, a lime oil, a macadamia nut oil, a mandarin oil, a marjoram oil, a myrrh oil, a myrtle oil, a neroli oil, an ocmea origanum oil, an orange oil, an oregano oil, a patchouli oil, a pepper oil, a peppermint oil, a petitgrain oil, a pimento berries oil, a pine oil, a pulegium mint oil, a rose oil, a rosemary oil, a rosewood oil, a sage oil, a sandalwood oil, a sea fennel oil, a sesame oil, a spearmint oil, a spikenard oil, a tea tree oil, a thyme oil, a vervail oil, a vetiver oil, a violet oil, a wheat oil, a wintergreen oil or a combination thereof.

3. The antimicrobial composition of claim 1, wherein the one or more essential oil is a Myrtus communis oil, Thymus capitatus oil, a Mentha pulegium oil, or a Eucalyptus cinerea oil.

4. The antimicrobial composition of claim 1, wherein the one or more cationic surfactant comprises at least one of an ionic liquid.

5. The antimicrobial composition of claim 4, wherein the at least one ionic liquid comprise an imidazolium-based surfactant, a benzimidazolium-based surfactant, a quaternary ammonium (QAC)-based surfactant, a pyridinium-based surfactant, a pyrrolidium-based surfactant, or a combination thereof.

6. The antimicrobial composition of claim 5, wherein the at least one ionic liquid comprises the imidazolium-based surfactant, the benzimidazolium-based surfactant, or a combination thereof.

7. The antimicrobial composition of claim 6, wherein the imidazolium-based surfactant comprises at least one of a 1-octyl-3-methylimidazolium salt, a 1,3-didecyl-2-methylimidazolium salt, a 1-decyl-3-methylimidazolium salt or a combination thereof.

8. The antimicrobial composition of claim 6, wherein the benzimidazolium-based surfactant comprises a 1,12-Bis((1-methylbenzimidazolium)-3-yl)dodecane salt.

9. The antimicrobial composition of claim 6, wherein the imidazolium-based surfactant compound and/or the benzimidazolium-based surfactant comprises one or more cleavable ester, ether, amide or hydroxyl functional group present in a hydrophobic chain.

10. The antimicrobial composition of claim 1, wherein the molarity to volume ratio of cationic surfactant to the essential oil is equal to or less than 15:1.

11. The antimicrobial composition of claim 1, comprising micelles have an average diameter of 1000 nm or less.

12. The antimicrobial composition of claim 1, comprising micelles have an average diameter of 100 nm or less.

13. The antimicrobial composition of claim 1, comprising a first population of micelles having a first average diameter and a second population of micelles having a second average diameter, wherein the first average diameter is different from the second average diameter.

14. The antimicrobial composition of claim 1, wherein the carrier is an aqueous solution.

15. The antimicrobial composition of claim 14, wherein the carrier lacks an organic solvent.

16. The antimicrobial composition of claim 14, being an emulsion.

17. The antimicrobial composition of claim 1, wherein the carrier is a solid or a gaz.

18. (canceled)

19. The antimicrobial composition of claim 18, being aerosolized and/or biodegradable.

20. (canceled)

21. (canceled)

22. (canceled)

23. (canceled)

24. (canceled)

25. (canceled)

26. A method of preventing microbial growth, reducing the viability of or inactivating microbes on a solid, in a liquid or in a gas, the method comprising contacting the antimicrobial composition of claim 1 with the solid, the liquid or the gas.

27. The method of claim 26, wherein a reduction of at least one log in the amount of the microbe is observed after contact with the antimicrobial composition.

28. (canceled)

29. (canceled)

30. (canceled)

31. (canceled)
